(19) United States
(12) Patent Application Publication
(10) Pub. No.: US 2019/0201578 A1
(43) Pub. Date: Jul. 4, 2019

(54) Biomaterial Implant

(71) Applicant: The Johns Hopkins University, Baltimore, MD (US)

(72) Inventors: Morgana M. Trexler, Baltimore, MD (US); Xiomara Calderon-Colon, Fulton, MD (US); Leslie H. Hamilton, Silver Spring, MD (US); Min Zhao, Sacramento, CA (US); Brian Reid, Sacramento, CA (US); Julia B. Patrone, Ellicott City, MD (US); Lance M. Baird, Los Angeles, CA (US)

(21) Appl. No.: 16/171,470

(22) Filed: Oct. 26, 2018

Related U.S. Application Data
(60) Provisional application No. 62/610,992, filed on Dec. 28, 2017.

(51) Int. Cl. A61L 27/24; A61L 27/44; A61F 9/00; A61L 27/56; A61K 9/00

(52) U.S. Cl. CPC A61L 27/24; A61L 27/44; A61F 9/0017; A61L 27/52; A61K 9/0051; A61L 2430/16; A61L 27/56

(57) ABSTRACT

A biomaterial implant may include a collagen membrane. The biomaterial implant may further include a plurality of nanoparticles embedded in the collagen membrane. Furthermore, at least one nanoparticle of the plurality of nanoparticles may include a polymer shell and a bio-active therapeutic agent encapsulated by the polymer shell.

### CROSS-REFERENCE TO RELATED APPLICATIONS

This application claims the benefit of U.S. Provisional Application No. 62/610,992 filed on Dec. 28, 2017, the entire contents of which are hereby incorporated herein by reference.

### STATEMENT REGARDING FEDERALLY SPONSORED RESEARCH OR DEVELOPMENT

This invention was made with Government support under contract number W81XWH-14-1-0542 awarded by the U.S. Department of the Army. The Government has certain rights in the invention.

### TECHNICAL FIELD

Exemplary embodiments of the present disclosure generally relate to a biomaterial implant that is configured to aid in the healing of an injury or disease.

### BACKGROUND

While eyes may account for only 0.1% of the frontal silhouette of a person, incidence of ocular injury has been shown to be high, particularly amongst military professionals and young children. Furthermore, ocular injuries are a leading cause of visual loss with some of the most common ocular injuries being to the cornea. Various drug delivery systems such as viscous ointments and polymeric hydrogels have been used in the past to treat cornea injuries. However, ointments and hydrogels suffer from the limitation of having to be reapplied.

### BRIEF SUMMARY

In one example embodiment, a biomaterial implant may be provided. The biomaterial implant may include a collagen membrane. The biomaterial implant may further include a plurality of nanoparticles embedded in the collagen membrane. Furthermore, at least one nanoparticle of the plurality of nanoparticles may include a polymer shell and a bio-active therapeutic agent encapsulated by the polymer shell.

In a further example embodiment, a method for preparing a biomaterial implant may be provided. The method may include applying an electric field influence to an acidic collagen solution positioned between metal plates and adding a buffer solution to the acidic collagen solution to form a collagen gel. The method may further include assembling a plurality of collagen gel layers and performing a dehydrothermal cross-link on the plurality of collagen gel layers to form a cross-linked collagen membrane. The method may even further include incorporating a plurality of nanoparticles into the cross-linked collagen membrane, wherein at least one nanoparticle of the plurality of nanoparticles comprises a polymer shell and a bio-active therapeutic agent encapsulated by the polymer shell.

### BRIEF DESCRIPTION OF THE DRAWING(S)

Having thus described example embodiments in general terms, reference will now be made to the accompanying drawings, which are not necessarily drawn to scale, and wherein:

FIG. 1 illustrates a biomaterial implant according to an example embodiment;

FIG. 2 illustrates a biomaterial implant implanted on a cornea according to an example embodiment;

FIG. 3 illustrates a therapeutic-containing nanoparticle of a biomaterial implant according to an example embodiment;

FIG. 4 illustrates a degradation profile and therapeutic release of a nanoparticle according to an example embodiment;

FIG. 5 illustrates a graphical representation of a therapeutic release from nanoparticles according to an example embodiment;

FIG. 6 illustrates a graphical representation of a therapeutic release from nanoparticles according to a further example embodiment;

FIG. 7 illustrates a block diagram of a method for preparing a biomaterial implant according to an example embodiment;

FIG. 8 illustrates scanning electron microscope (SEM) images of collagen membranes according to an example embodiment

FIG. 9 illustrates time lapse wound current data for both a shallow (normal epithelial) wound and a deep (stromal) wound according to an example embodiment;

FIG. 10 illustrates time lapse wound healing data for both a shallow (normal epithelial) wound and a deep (stromal) wound according to a further example embodiment;

FIG. 11 illustrates the effect of aminophylline on current in a wound according to an example embodiment;

FIG. 12 illustrates the effect of the aminophylline in a wound on healing time according to a further example embodiment;

FIG. 13 illustrates the percentage of wound healing as a function of time for corneas treated with aminophylline according to an example embodiment;

FIG. 14 illustrates factors employed in a method of preparing the biomaterial implant, and resulting implant properties, according to an example embodiment; and

FIG. 15 illustrates properties and morphology of a biomaterial implant prepared under various conditions, according an example embodiment.

### DETAILED DESCRIPTION

Some example embodiments now will be described more fully hereinafter with reference to the accompanying drawings, in which some, but not all example embodiments are shown. Indeed, the examples described and pictured herein should not be construed as being limiting as to the scope, applicability or configuration of the present disclosure. Rather, these example embodiments are provided so that this disclosure will satisfy applicable legal requirements. Like reference numerals refer to like elements throughout. Furthermore, as used herein, the term “or” is to be interpreted as a logical operator that results in true whenever one or more of its operands are true.

Example embodiments herein are directed to a biomaterial implant that is configured to aid in healing of wounded or damaged tissue, such as an ocular wound, injury, or disease. In this regard, the biomaterial implant of example embodiments contained herein may be configured to significantly improve the healing rate of a wounded or damaged tissue, such as an injury to the eye or the like, or a disease. The biomaterial implant may include a collagen composite with a plurality of nanoparticles embedded or incorporated therein. The nanoparticles may each include a polymeric shell where each polymer shell is configured to encapsulate a therapeutic or pharmaceutical agent. The therapeutic or pharmaceutical agent may be configured to be released or expelled from the polymer shell over a predetermined period of time into a wound. The biomaterial implant therefore may allow for a controlled and extended release of a bio-active therapeutic agent over a predetermined time frame to the disease or the wound to aid in the healing of the injury and reduce patient recovery time without limitations such as having to be constantly reapplied by a user.

Due to rapid removal of foreign materials from cornea tissues and limitations of viscous ointments and polymeric hydrogels, a biomaterial implant enabling the extended release of a bio-active therapeutic agent to tissue that has been inflicted with an injury, such as an ocular injury, during a healing process may significantly improve the healing rate of the tissue and therefore reduce patient recovery time. The biomaterial implant described herein may include a collagen composite with a plurality of nanoparticles embedded or incorporated therein. Each nanoparticle may provide for a polymeric encapsulate that is configured to slowly erode or degrade over time. The biomaterial implant may therefore allow for a controlled and extended release of the bio-active therapeutic agent over a predetermined time frame (e.g., 1-8 days) to an injury, such as, an ocular injury, or a disease without having to be constantly reapplied by a user.

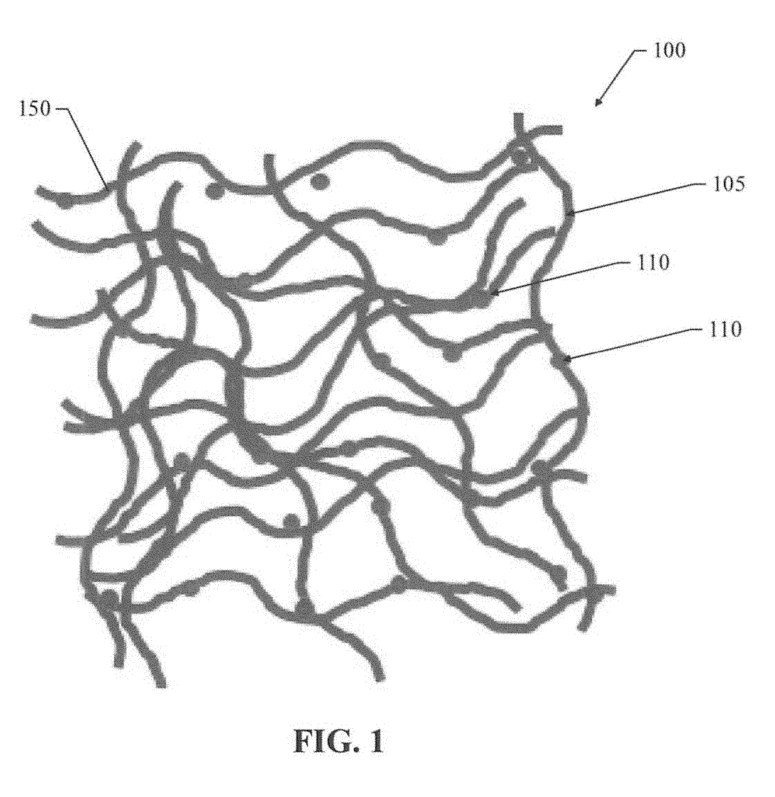

According to example embodiments contained herein and as shown in FIG. 1, a biomaterial implant material 100 may be provided. The biomaterial implant 100 may be an inclusive, point-of-care device that has integrated pharmaceutical delivery and healing benefits. In other words, the biomaterial implant 100 may include a pharmaceutical or therapeutic agent that is configured to be applied to and heal a tissue and does not have to be constantly reapplied to the tissue. In this regard, the biomaterial implant 100 may include a collagen membrane 105 with nanoparticles (or nanocarriers) 110 incorporated or embedded therein. The nanoparticles 110 may encapsulate a therapeutic or pharmaceutical agent to aid in accelerating the treatment and healing of injured or diseased tissue, thereby resulting in reduced patient recovery time. For example, the biomaterial implant 100 may be configured to be implanted at a wound site following an injury. Example wounds that may benefit from the biomaterial implant 100 may arise from a blast, blunt, burn, ballistic, or penetrating injury. In other words, the biomaterial implant material 100 may be applied to an injured cornea, skin, chronic wounds (e.g., diabetic or pressure ulcers), or the like.

In some cases, the biomaterial implant 100 may be configured or designed to mimic a topography of the wound it is intended to treat. In this regard, when the biomaterial implant 100 is an ocular implant configured to treat a cornea, for example, the biomaterial implant 100 may be configured in a manner that mimics the topography of the cornea. The biomaterial implant 100 may include or incorporate a plurality of the nanoparticles 110 that enable an extended release of a therapeutic or pharmaceutical agent. These nanoparticles 110 may be, for example, solid lipid nanoparticles, gelatin nanoparticles, or polymer nanoparticles (e.g., poly(lactic-co-glycolic acid) (PLGA) nanoparticles). The nanoparticles 110 may be configured to deliver therapeutic or pharmaceutical agents over a predetermined period of time (e.g., 48-72 hours, 1-8 days, etc.).

In this regard, the biomaterial implant 100 described herein may be configured with a biomimetic chemistry, nanotopography, and a drug or therapeutic agent delivery functionality that may improve wound healing, in particular ocular wound healing, or disease treatment. For example, the ocular wound healing may be improved by: 1) providing biochemical and biophysical signals or cues for enhanced cell migration, differentiation, and proliferation; and 2) delivering chemical bioelectric modulators (e.g., chloride) for enhancing wound electric fields.

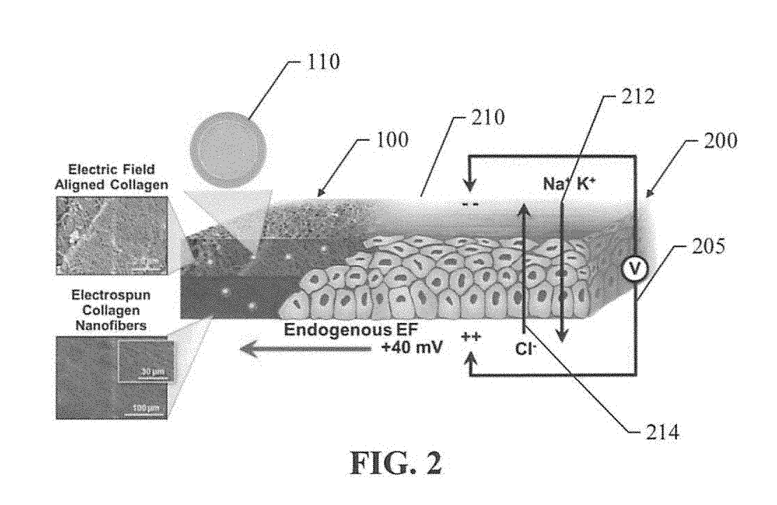

FIG. 2 illustrates an example embodiment of the biomaterial implant 100 implanted on a cornea 200 of an eye. As shown in FIG. 2, a voltage 205 of the ocular injury (e.g., cornea injury) may initiate repair of a damaged cornea tissue 210. This voltage 205 may be generated by epithelial disruption of sodium/potassium (Na⁺/K⁺) adenosine triphosphatase (ATPase) pumps 212 and thus generates a lateral electric field. Wound electric fields may affect cell migration, division, proliferation, and nerve sprouting.

In this regard, electric fields may occur at a wound in the cornea, and a wound in the corneal epithelium may generate a transepithelial potential difference of approximately 25-45 mV. The electrical potential may be generated by the epithelium of the cornea pumping chloride from the stroma of the cornea out to tears and sodium from the tears to the stroma. Furthermore, close or narrow connections between epithelial cells of the cornea may form a barrier of high electrical resistance thereby maintaining the transepithelial potential difference. An injury or wound to the epithelium of the cornea may disrupt this barrier and short-circuit the transepithelial potential difference locally at the wound or injury. Positive potential under the surrounding intact epithelium (i.e., part of the epithelium that is not injured) may drive ion currents into the wound producing endogenous wound electric currents and a lateral electric field projecting from around the wound into the wound center.

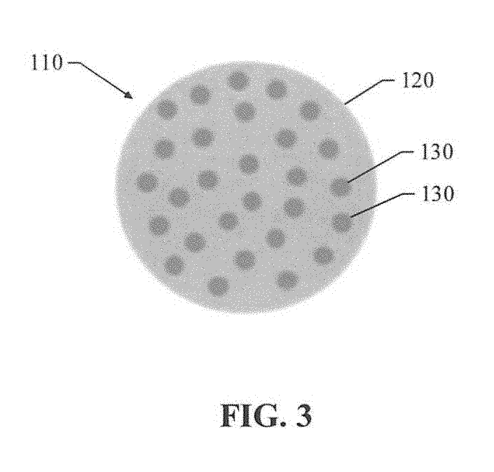

Furthermore, in the case of the damaged cornea tissue 210, an endogenous wound electric field (EF), which arises due to active ion transport in an intact cornea epithelium surrounding the damaged cornea tissue 210, may signal cells to begin the healing process of the cornea 200. This endogenous wound EF may be a vector, which constantly points towards the wound center, and may act as a mechanism for guiding new cells into the damaged cornea tissue 210 to aid with wound healing. Certain therapeutic or pharmacological agents (e.g., aminophylline as discussed further herein), which may be encapsulated by the nanoparticle 110 as discussed below, may modulate the wound EF. When introduced into the damaged cornea tissue 210, these therapeutic or pharmacological agents may change the magnitude of the EF by increasing or decreasing cyclic adenosine monophosphate (cAMP) levels, thereby causing a healing rate to increase or decrease depending on whether the therapeutic or pharmacological agent is configured to either stimulate or inhibit the wound EF. Accordingly, as further discussed below, the biomaterial implant 100 according to example embodiments herein may include a bio-active therapeutic agent 130 (see FIG. 3) that is encapsulated in the nanoparticle 110 and is configured to increase the wound electric signal thereby increasing the healing rate to the ocular wound. In this regard, the bio-active therapeutic agent 130 may be configured to increase cAMP levels to enhance chloride (CF) pumping 214 to the damaged cornea tissue 210.

In some example embodiments, the biomaterial implant 100 may be configured to closely mimic an in vivo cornea environment such as by exhibiting transparency (greater than 75% to maintain vision), mechanical (suture) strength, and properties important for high performance corneal implants. Furthermore, incorporation of the bio-active therapeutic or pharmaceutical agent 130 into the biomaterial implant 100 may allowed tailored, sustained delivery of bioelectric modulators that may stimulate cell responses related to the healing process. Accordingly, the biomaterial implant 100 may be configured with a tailored nanotopography that has the ability to deliver endogenous wound electric field-enhancing molecules that will encourage and accelerate the wound healing process.

In accordance with example embodiments herein, the biomaterial implant 100 (e.g., collagen membrane) may have a thickness from about 100 microns to about 600 microns. In other embodiments, for example, the biomaterial implant 100 may have a thickness from about 150 microns to about 500 microns. In further embodiments, for instance, the biomaterial implant 100 may have a thickness from about 250 microns to about 500 microns. In some embodiments, for example, the biomaterial implant 100 may have a thickness from about 300 microns to about 500 microns. In certain embodiments, for instance, the biomaterial implant may have a thickness of about 500 microns. As such, in certain embodiments, the biomaterial implant 100 may have a thickness in a range of approximately 100, 150, 200, 250, 300, 350, 400, 450, and 500 microns and/or 600, 590, 580, 570, 560, 550, 540, 530, 520, 510, and 500 microns (e.g., about 250-500 microns, about 300-550 microns, etc.).

In certain embodiments, the thickness may be substantially the same as a corneal thickness (e.g., 500 microns). As such, in some embodiments, the biomaterial implant 100 may be configured to be sutured to an eye (e.g., by being shaped as a disk via, for example, a biopsy punch or a mold having the curvature of the eye). In this regard, the biomaterial implant 100 may be of an appropriate thickness for use in a full human corneal replacement (e.g., about 500 microns), a partial human corneal replacement (e.g., about 250 microns), a full animal (e.g., rabbit) corneal replacement (e.g., about 300 microns), a partial animal (e.g., rabbit) corneal replacement (e.g., about 150 microns), or the like.

In accordance with example embodiments herein, the biomaterial implant 100 (e.g., collagen membrane) may have a suture strength that enables a user implanting the device to easily manipulate the biomaterial implant 100 while successfully suturing the bioimplant material 100 to the wound. In this regard, a suture strength of the biomaterial implant 100 may range from about 0.09 mN/micron to about 4 mN/micron. In other embodiments, for example, the biomaterial implant 100 may have a suture strength from about 0.1 mN/micron to about 3 mN/micron. In further embodiments, for instance, the biomaterial implant 100 may have a suture strength from about 1 mN/micron to about 2 mN/micron. In some embodiments, for example, the biomaterial implant 100 may have a suture strength from about 0.09 mN/micron to about 0.4 mN/micron. As such, in certain embodiments, the biomaterial implant 100 may have a suture strength range of approximately 0.09, 0.1, 0.2, 0.3, 0.4, 0.5, 0.6, 0.7, and 0.8, 0.9 mN/micron and/or 1.0, 1.5, 2.0, 2.5, 3.0, 3.5, and 4.0 mN/micron (e.g., about 0.09-1.0 mN/microns, about 0.4-2 mN/microns, etc.).

As further shown in FIG. 1, the collagen membrane 105 may include a plurality of fibers 150. In some cases, the fibers 150 may be substantially aligned in a single direction within the collagen membrane 105. In this regard, the fibers 150 may be arranged in parallel thereby resulting in minimal light scattering, which maintains clarity in a cornea of an eye, for example. Furthermore, when used to treat an ocular wound, the fibers 150 may be collagen fibers. According to certain embodiments, for example, a diameter of each fiber 150 may be from about 20 nm to about 300 nm. In other embodiments, for instance, the fibers 150 may have a diameter from about 30 nm to about 200 nm. In further embodiments, for example, the fibers 150 may have a diameter from about 40 nm to about 100 nm. In some embodiments, for instance, the fibers 150 may have a diameter from about 50 nm to about 90 nm. As such, in certain embodiments, the fibers 150 may have a diameter range of approximately 20, 21, 22, 23, 24, 25, 30, 35, 40, 45, 50, 60, or 70 nm and/or 55, 60, 65, 70, 75, 80, 85, 90, 95, 100, 150, 200, 250, or 300 nm (e.g., about 20-300 nm, about 40-70 nm, etc.).

Furthermore, nanoparticles 110 may be integrated, embedded, or otherwise incorporated into the collagen membrane 105 of biomaterial implant 100. In this regard, to incorporate the nanoparticles 110, the collagen membrane 105 may be soaked in a solution that includes the nanoparticle 110. In some cases, however, the nanoparticles 110 may be pipetted on the collagen membrane 105 to allow the nanoparticles to absorb and crosslink on the collagen membrane 105.

This integration of the nanoparticles 110 into the collagen membrane 105 may be accomplished by covalent functionalization. In this regard, the functionalization of the nanoparticles 110 into the collagen membrane 105 may be accomplished by: 1) physical crosslinking or entanglement; 2) chemical cross-linking that relies on irradiation by ultra-violet (UV) light or addition of chemical cross-linking agents; 3) chemical techniques; or 4) formation of ionic bonds or the like. In example embodiments where the nanoparticles are PLGA nanoparticles, the chemical crosslinking using UV irradiation accomplishes the integration by exploiting amino (—NH2) in collagen and carboxyl (—COOH) functional groups of PLGA to ensure the covalent functionalization.

FIG. 3 illustrates an example embodiment of the nanoparticle 110 of the biomaterial implant 100. The nanoparticle 110 described herein may have diameters of about 50 nanometers to 300 nanometers. Accordingly, the nanoparticle 110 may have a diameter in a range of approximately 50, 55, 60, 65, 70, 75, 80, 85, 90, 95, or 100 nanometers and/or 110, 115, 120, 125, 130, 135, 140, 145 150, 155, 160, 165, 170, 175, 180, 185, 190, 195, 200, 250, or 300 nanometers (e.g., about 70-100 nanometers, about 80-120 nanometers, etc.). In embodiments, where the biomaterial implant 100 is used for treating an ocular injury, the nanoparticle 110 may have a predefined diameter tailored such that the nanoparticle 110 does not scatter light (e.g., about 75-125 nanometers). As a result, a cornea of an eye, for example, may maintain or sustain clarity (i.e., avoid haziness). Because haziness of the cornea may be avoided, the nanoparticle 110 may enable efficient and effective treatment of the wound.

As shown in FIG. 3, each nanoparticle 110 may include a polymer shell 120. The polymer shell 120 may be configured to encapsulate the bio-active therapeutic agent 130 that is configured to assist in healing the ocular injury. Furthermore, the polymer shell 120 may include any polymer that is bio-compatible with the wound to be treated. In some cases, the polymer may be a hydrophobic polymer. Furthermore, the polymer may be configured to degrade in a presence of water or an aqueous environment or solution over a predetermined period of time. A polymer configured to slowly degrade may allow for an extended and controlled release of the encapsulated bio-active therapeutic agent 130 through the collagen membrane 105 to the wound, injury, or targeted area of the body.

In some cases, the polymer may be PLGA. PLGA is biocompatible with ocular tissues and has a molecular weight that may be tailored, as desired, in order to change a length of a release time of the bio-active therapeutic agent 130, as desired. In this regard, higher molecular weight polymers (e.g., 38,000-54,000 g/mol) exhibit a slower release rate, and lower molecular weight polymers (e.g., 7,000-17,000 g/mol) exhibit a faster release rate. The ability to alter the release rate of the bio-active therapeutic agent 130 by using different molecular weight polymers enables the release rate of the therapeutic agent 130 to be tuned by simply constructing nanoparticles composed of different molecular weight PLGA polymers. However, as discussed above, interaction between the polymer shell 120 and the therapeutic agent 130 may also influence the release rate of the therapeutic agent 130 into the wound. Furthermore, the specific therapeutic agent 130 used may negate any effect the molecular weight has on the release rate. In other embodiments, the polymer nanoparticles may be created using polycaprolactone or polylactic acid.

PLGA may undergo degradation through hydrolysis of its ester linkages in the presence of water or an aqueous solution thus allowing for extended release of the therapeutic agent 130. Specifically, a ratio of lactic acid to glycolic acid in PLGA may be adjusted to achieve a desired degradation rate of the polymer shell 120. In some cases, the ratio of lactic acid to glycolic acid may be about 50:50. However, in other cases, the ratio of lactic acid to glycolic acid may be about any of 10:90, 20:80, 30:70, 40:60, 60:40, 70:30, 80:20, or 90:10.

An increase in the glycolic acid component of PLGA may make the polymer shell 120 more hydrophilic. In this regard, by having a PLGA polymer with a much higher concentration of glycolic acid, the polymer shell 120 may have an increased ability to entrap the therapeutic agent 130. However, the higher concentration of glycolic acid may lead to a much more hydrophilic polymer shell 120 thus causing a faster release rate of the therapeutic agent 130. By having a PLGA polymer with a much lower concentration of glycolic acid, the polymer shell 120 may have a lower ability to entrap the therapeutic agent 130. However, the polymer shell 120 would be more hydrophobic thus causing a slower release rate of the polymer shell 120. Based on the specific components of the polymer, the entrapment efficiency of the polymer 120 may range from about 0.1-10% (m/m) bio-active therapeutic agent entrapment efficiency. For example, when the ratio of lactic acid to glycolic acid in PLGA is about 50:50, the entrapment efficiency of the polymer shell 120 may be about 1% (m/m), which yields enough bio-active therapeutic agent 130 to provide a significant and desirable biological response in the wound.

As further shown in FIG. 3, the nanoparticle 110 may also include the therapeutic agent 130 that may be encapsulated by the polymer shell 120. The therapeutic agent 130 may be any therapeutic agent 130 that is configured to be delivered to a target area of a body to, for example, aid or assist in the healing of the wound, such as an ocular wound. As discussed above, in relation to a cornea injury, the therapeutic agent 130 may be a therapeutic agent that is configured to stimulate the wound EF to increase a healing rate of the wound. In some cases, the therapeutic agent 130 may be a water-soluble therapeutic agent. Furthermore, in cases where the polymer shell 120 is a hydrophobic polymer shell 120, the therapeutic agent 130 may be a hydrophilic therapeutic agent to enable the therapeutic agent 130 to partition out of the polymer shell 120 through the collagen membrane 105 into the aqueous environment of the eye. Of course, it should be understood that the therapeutic agent 130 may be hydrophobic or hydrophilic and chosen based on the specific polymer shell 120 used or wound, injury, or disease to be treated.

In embodiments, where the wound is an ocular wound, the therapeutic agent 130 may be aminophylline. Aminophylline is a non-specific phosphodiesterase inhibitor that is configured to stimulate the wound EF by elevating cyclic adenosine monophosphate levels, which increase the current in the wound by increasing chloride transport (see FIG. 2). Specifically, when the wound is a cornea wound, aminophylline may increase the current in the wound by increasing chloride transport from an aqueous humor to a tear side of the cornea. Furthermore, aminophylline is a hydrophilic therapeutic agent with a log partition (log P) coefficient value of −3.03. The log P coefficient value of aminophylline means that there is a relatively large concentration based driving force for the therapeutic agent 130 to partition out of the polymer shell 120 of the nanoparticle 110 into the eye (e.g., an aqueous environment of the cornea). It should be understood that, in other embodiments, the bio-active therapeutic agent 130 may be tailored to a specific injury or disease (e.g., the ocular wound, breast cancer, etc.). Accordingly, in some cases where the bio-active therapeutic agent 130 is tailored to treat breast cancer, the bio-active therapeutic agent 130 may be or include fulverstant.

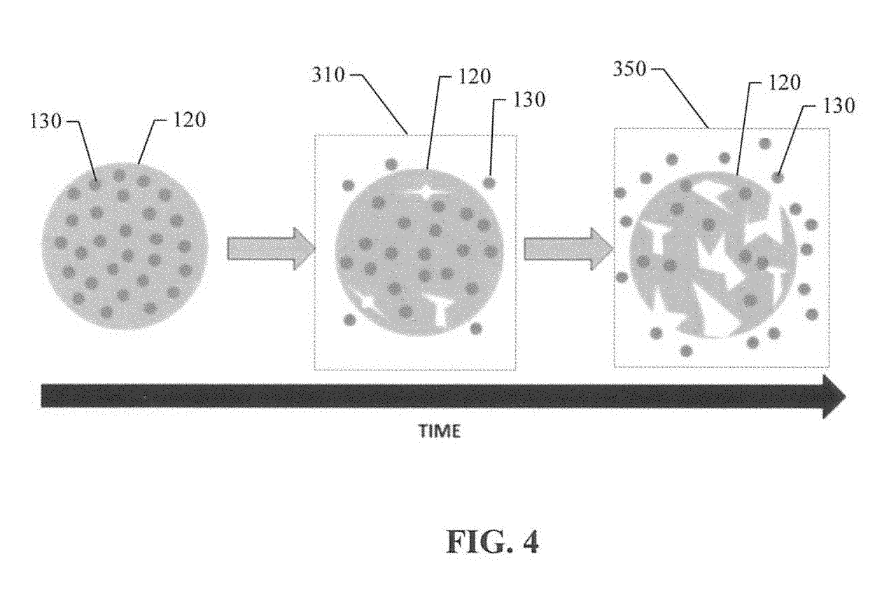

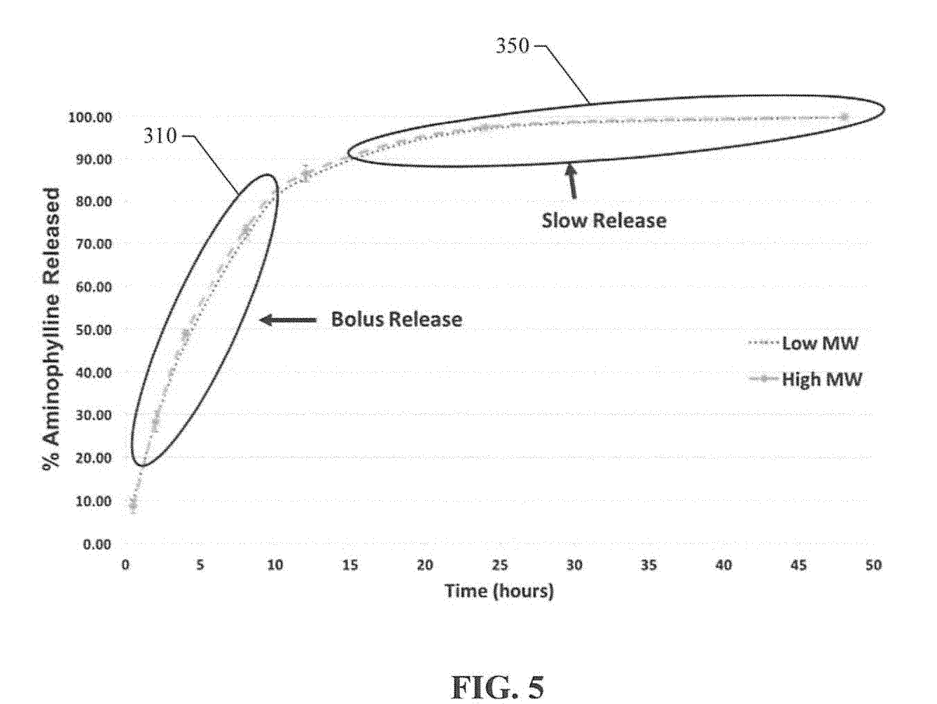

FIGS. 4 and 5 illustrate an example embodiment of a degradation profile or a release of the bio-active therapeutic agent 130 from the nanoparticle 110 of the biomaterial implant 100. As mentioned above, the polymer shell 120 may be configured to degrade at a predetermined rate in order to enable the release of the bio-active therapeutic agent 130 through the collagen membrane 105 into the wound. In this regard, the polymer shell 120 may include a degradation profile such that the bio-active therapeutic agent 130 is completely released into the wound in a predetermined period of time (e.g., 36-72 hours, 1-8 days, etc.). In other words, the polymer shell 120 may be tailored such that the therapeutic agent is exhausted from the shell in about 1-8 days. Accordingly, in some embodiments, the polymer shell 120 may be configured such that the therapeutic agent is exhausted or expelled from the polymer shell 120 by approximately 36, 40, 48, or 72 hours or approximately 4, 5, 6, 7, or 8 days (e.g., about 40-70 hours, about 48-72 hours, etc.).

The degradation profile of the polymer shell 120 may include that the bio-active therapeutic agent is released at predefined amounts in certain phases. In this regard, the degradation or release profile may include a bolus phase 310 and a slow release phase 350. In other words, the degradation profile may be a biphasic structure such that there is a bolus release phase 310 of the bio-active therapeutic agent 130 and a slow release phase 350 of the bio-active therapeutic agent 130. In accordance with some example embodiments, the bolus release phase 310 may be an initial burst release of the bio-active therapeutic agent 130 that happens over about 5-20 hours as a result of exposure of the nanoparticle 110 to an aqueous environment of the wound. In other words, the bolus release phase 310 may originate from osmotic pumping of the bio-active therapeutic agent 130 from a surface of the polymer shell 120 (e.g., hydrophilic bio-active therapeutic agent 130 out of the hydrophobic polymer shell 120). The slow release phase 350 may be an extended, steady release of the bio-active therapeutic agent 130 that is configured to occur after the bolus release phase 310 and happen over about 10-72 hours after the bolus release phase 310. The slow release phase 350 may be caused by diffusion of the bio-active therapeutic agent 130 through a matrix or pores of the polymer shell 120 or bulk hydrolysis of the polymer shell 130. Furthermore, the bolus release phase 310 may last from about 5-20 hours and the slow release phase 350 may last from about 24-70 hours. Additionally, the bolus release phase 310 may include a release of about 50-80% of the bio-active therapeutic agent 130 over about 5-20 hours and the slow release phase 310 may include a release of about 20-50% of the bio-active therapeutic agent 130 over 24-70 hours.

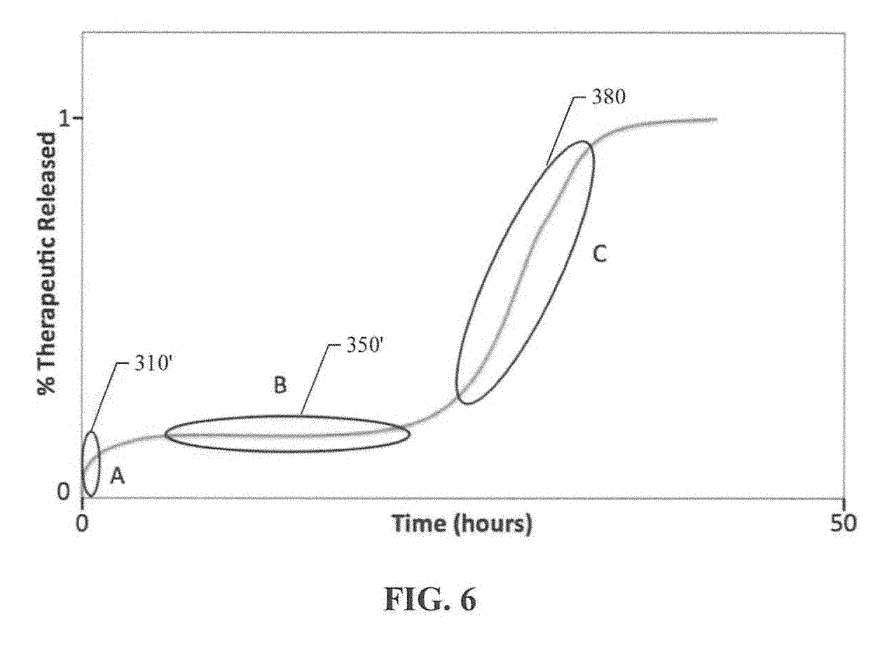

FIG. 6 illustrates an example embodiment of a triphasic degradation profile of the nanoparticle 110 of the biomaterial implant 100. In some example embodiments, the degradation profile of the polymer shell 120 may be a triphasic structure such that there is an initial bolus phase 310′ (A), a slow release phase 350′ (B), and finally a fast release phase 380 (C). In embodiments where the degradation profile is triphasic, the bolus release phase 310′ may be an initial burst release of the bio-active therapeutic agent 130 that is configured to happen over about 5-15 hours from the exposure of the nanoparticle 110 to the aqueous environment of the wound. The slow release phase 350′ may be an extended, steady release of the bio-active therapeutic agent that is configured to happen after the bolus release phase 310′ and over about 10-40 hours. The fast release phase 380 may follow the slow release phase 350′ and be a burst release that happens over about 5-15 hours. Furthermore, the bolus release phase 310′ may include a release of about 10-20% of the bio-active therapeutic agent. The slow release phase 350′ may include a release of about 10-20%, and the fast release phase 380 may include a release of about 60-80% of the bio-active therapeutic agent.

In some example embodiments, the phases of the degradation profile may be caused by or related to the interaction of the polymer shell 120 with the bio-active therapeutic agent 130. In this regard, the bolus release phase 310 may be caused by an initial burst of any surface bound bio-active therapeutic agents 130 out of the polymer shell 120. In other words, the bolus release phase 310, 310′ may originate from osmotic pumping of the bio-active therapeutic agent (e.g., hydrophilic bio-active therapeutic agent). The slow release phase 350, 350′ may be caused by diffusion of the bio-active therapeutic agent 130 through a matrix or pores of the polymer shell 120. In embodiments that include a fast release phase 380, the fast release phase 380 may be caused by bulk hydrolysis of the polymer shell 120.

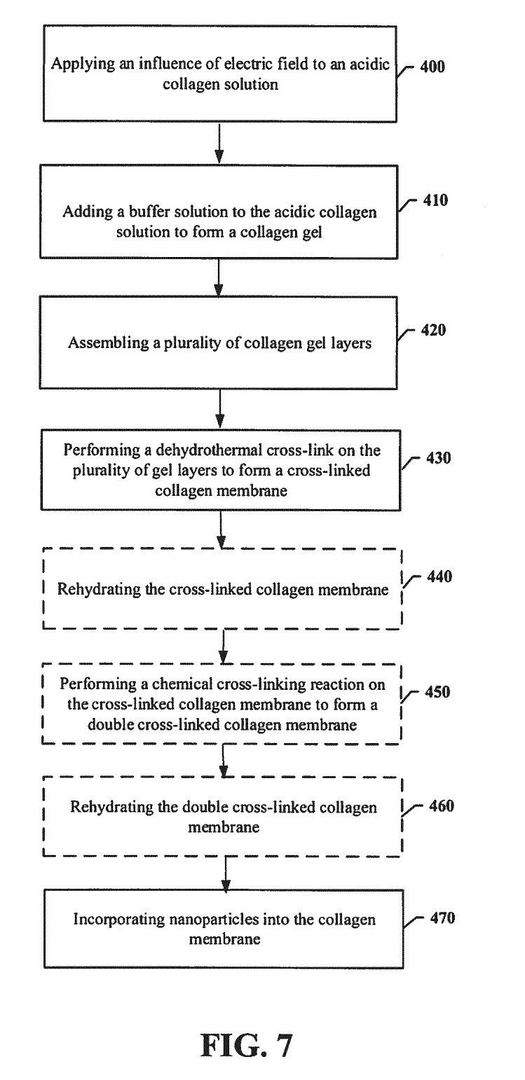

FIG. 7 illustrates a method of preparing the biomaterial implant 110 described herein. As shown in FIG. 7, the method may include applying an influence of an electric field to an acidic collagen solution positioned between metal plates at operation 400, adding a buffer solution to the acidic collagen solution to form a collagen gel at operation 410, assembling a plurality of collagen gel layers at operation 420, performing a dehydrothermal cross-link on the plurality of collagen gel layers to form a cross-linked collagen membrane at operation 430, and incorporating nanoparticles described herein into the cross-linked collagen membrane at operation 470. In some cases, the method may also include the optional steps of rehydrating the cross-linked collagen membrane at operation 440, performing a chemical cross-linking reaction on the cross-linked collagen membrane to form a double cross-linked collagen membrane at operation 450, or rehydrating the double cross-linked collagen membrane at operation 460.

As mentioned above, the method may include the initial step of applying an influence of an electric field to an acidic collagen solution positioned between metal plates, such as two metal plates, at operation 400. In some cases, the collagen solution may comprise dissolving collagen powder (e.g., Type I bovine collagen), in, for example, 1,1,1,3,3,3-hexafluoro-2-propanol (HFP). In some cases, the collagen solution may be poured onto a metal mold inside a Petri dish, covered, and placed between the two plates.

In accordance with example embodiments, the collagen concentration may include from about 5 mg/ml collagen to about 20 mg/ml collagen. In other embodiments, for instance, the collagen concentration may include from about 6 mg/ml collagen to about 15 mg/ml collagen. In further embodiments, for example, the collagen concentration may include from about 7 mg/ml collagen to about 10 mg/ml collagen. In some embodiments, for instance, the collagen concentration may include about 8 mg/ml collagen. As such, in certain embodiments, the collagen concentration may include the range of approximately 5, 6, 7, and 8 mg/ml collagen and/or 20, 18, 15, 12, 10, 9, and 8 mg/ml collagen (e.g., about 7-15 mg/ml collagen, about 7-9 mg/ml collagen, etc.).

Moreover, in certain embodiments, the metal plates may be stainless steel plates. In some cases, the metal plates may be capacitor plates. Additionally, the metal plates may include an upper plate and a lower plate. The upper plate and the lower plate may be flat, horizontal plates situated substantially parallel to each other, and an electric field may be formed by transmitting energy from the upper plate to the lower plate. When energy is transmitted from the upper plate through the acidic collagen solution to the lower plate, some energy may have an influence on the acidic collagen solution, and some energy may be received by the lower plate.

In accordance with an example embodiment, applying the influence of the electric field to the collagen solution positioned between the metal plates may include applying a voltage between metal plates for a time period of about 1 hour to about 15 hours. In other embodiments, for instance, applying the influence of the electric field to the collagen solution positioned between the metal plates may include applying a voltage between metal plates for a time period of about 5 hours to about 12 hours. In further embodiments, for example, applying the influence of the electric field to the collagen solution positioned between the metal plates may include applying a voltage between metal plates for a time period of about 6 hours to about 10 hours. In certain embodiments, for instance, applying the influence of the electric field to the collagen solution positioned between the metal plates may include applying a voltage between metal plates for a time period of about 8 hours. As such, in certain embodiments, applying the influence of the electric field to the collagen solution positioned between the metal plates may include applying a voltage between metal plates for a time period with a range of approximately 1, 2, 3, 4, 5, 6, 7, and 8 hours and/or 15, 14, 13, 12, 11, 10, 9 and 8 hours (e.g., about 3-12 hours, about 7-9 hours, etc.).

According to certain embodiments, applying the influence of the electric field to the collagen solution positioned between the metal plates may include applying a voltage at an electrode distance (i.e., distance between the metal plates) from about 1 cm and about 5 cm. In other embodiments, for instance, applying the influence of the electric field to the collagen solution positioned between the metal plates may include applying a voltage at an electrode distance from about 2 cm and about 4 cm. In further embodiments, for example, applying the influence of the electric field to the collagen solution positioned between the metal plates may include applying a voltage at an electrode distance from about 2 cm and about 3 cm. In certain embodiments, for instance, applying the influence of the electric field to the collagen solution positioned between the metal plates may include applying a voltage at an electrode distance of about 1 cm. As such, in certain embodiments, applying the influence of the electric field to the collagen solution positioned between the metal plates may include applying a voltage at an electrode distance in a range of approximately 1, 2, 3, 4, and 5 cm and/or 5, 4.5, 4, 3.5, 3, 2.5, 2, 1.5, and 1 cm (e.g., about 1-3 cm, about 1-4 cm, etc.).

In certain embodiments, for example, applying the influence of the electric field to the collagen solution positioned between the metal plates may include applying a voltage from about 1 V to about 15 V. In other embodiments, for instance, applying the influence of the electric field to the collagen solution positioned between the metal plates may include applying a voltage from about 4 V to about 12 V. In further embodiments, for example, applying the influence of the electric field to the collagen solution positioned between the metal plates may include applying a voltage from about 5 V to about 10 V. In some embodiments, for instance, applying the influence of the electric field to the collagen solution positioned between the metal plates may include applying a voltage of about 5 V. In other embodiments, for example, applying the influence of the electric field to the collagen solution positioned between the metal plates may include applying a voltage of about 10 V. As such, in certain embodiments, applying the influence of the electric field to the collagen solution positioned between the metal plates may include applying a voltage in a range of approximately 1, 2, 3, 4, 5, 6, 7, 8, 9, and 10 V and/or 15, 14, 13, 12, 11, 10, 9, 8, 7, 6, and 5 V (e.g., about 5-12 V, about 9-10 V, etc.). However, it should be understood that the electrode distance and the applied voltage may vary depending on the desired electric field. For example, an electrode distance of 1 cm and an applied field of either 5 V or 10 V may produce an electric field of 5 V/cm and 10 V/cm respectively.

Moreover, in some embodiments, for instance, the collagen solution may be exposed to the influence of the electric field for a given period of time at a temperature from about 15° C. to about 45° C. In other embodiments, for example, the collagen solution may be exposed to the influence of the electric field for a given period of time at a temperature from about 20° C. to about 40° C. In certain embodiments, for instance, the collagen solution may be exposed to the influence of the electric field for a given period of time at a temperature of about 23° C. In further embodiments, for example, the collagen solution may be exposed to the influence of the electric field for a given period of time at a temperature of about 37° C. As such, in certain embodiments, the collagen solution may be exposed to the influence of the electric field for a given period of time at a temperature in a range of approximately 15, 18, 20, 21, 22, 23, 25, 30, 35, and 37° C. and/or 45, 43, 40, 39, 38, 37, 35, 30, 25, and 23° C. (e.g., about 23-37° C., about 21-35° C., etc.).

It should be understood that the collagen concentration, voltage applied, plate distance, temperature used, etc. may all be tailored in order to achieve a desired fiber diameter size and arrangement of the biomaterial implant 100. In this regard, the method for preparing the biomaterial implant 100 may allow for tailoring of collagen membrane thickness, fiber diameter, fiber density, thickness-normalized transparency, and suture strength. In this regard, for example, the following conditions of 7.75 wt. % collagen or 15.5 wt. % collagen, 0.5 mL/hr flow rate or 1.0 mL/hr flow rate, 15 kV applied voltage or 20kV applied voltage, and 10 cm collector distance or 12 cm collector distance may result in a fiber diameter of about 20 nm to 300 nm.

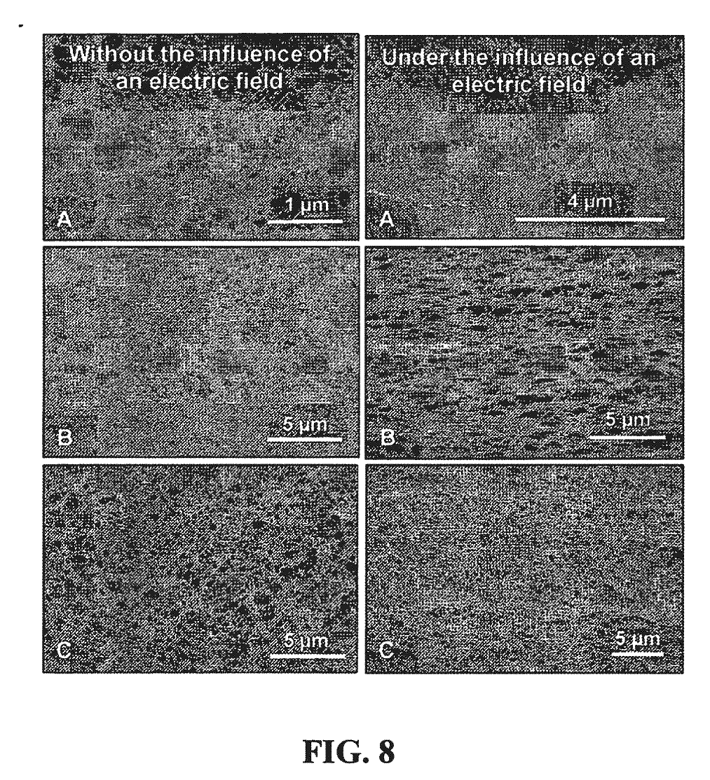

Additionally, applying the influence of the electric field to the collagen solution may result in different arrangement of collagen fibers 150 in the collagen membrane 105 than those collagen fibers that appear in conventional collagen materials. For example, FIG. 8 illustrates SEM images of collagen membranes 105 according to an example embodiment. As shown in FIG. 8, which illustrates a top view (A), first side view (B), and second side view (C) of the biomaterial implant 100, the influence of the electric field on the collagen solution resulted in a different arrangement of collagen fibers 150 than the control that was not exposed to the influence of the electric field.

After applying the electric field at step 400, a buffer solution may be added, at step 410, to the acidic collagen solution to form a collagen gel. As the collagen solution is influenced by the energy, a buffer may cause a change in the collagen solution pH from an acidic pH to a neutral pH. In this regard, by controlling the pH of the collagen solution, the charges along the collagen molecules may be controlled. During the gelation, collagen molecules may begin to assemble into a network structure that leads to the formation of a gel-like material. In some embodiments, as shown in step 420 for instance, a plurality of collagen gels may be assembled together into a plurality of collagen gel layers.

At step 430, the plurality of collagen gel layers may then be dehydrothermally cross-linked to form a collagen membrane that includes a plurality of collagen gel layers, and then at step 440, the cross-linked collagen membrane may be rehydrated. In accordance with certain embodiments, for instance, dehydrothermal cross-linking and rehydrating the membrane at least once may improve the membrane's mechanical properties (e.g., by controlling the humidity, temperature, and time). In some embodiments, for example, the membrane may be chemically cross-linked and rehydrated at least twice (see steps 450 and 460 of FIG. 7). According to certain embodiments, for instance, the collagen cross-linkers may comprise at least one of 1-ethyl-3-(3 dimethyl-aminopropyl)-1-carbodiimide hydrochloride (EDC), N-hydroxysuccinimide (NHS), riboflavin, 2-Hydroxy-4′-(2-hydroxyethoxy)-2-methylpropiophenone, or any combination thereof.

Thus, according to example embodiments herein, a biomaterial implant enabling the extended release of a bio-active therapeutic agent to a disease, wound, or injury, such as an ocular injury, during a healing process may be provided. The biomaterial implant described herein may include a collagen composite with a plurality of nanoparticles embedded or incorporated therein. Each nanoparticle may provide for a polymeric encapsulate that is configured to slowly erode or degrade over time. The biomaterial implant may therefore allow for a controlled and extended release of a bio-active therapeutic agent over a predetermined time frame (e.g., 48-72 hours, 1-8 days, etc.) to an ocular injury.

The following examples are provided to enable one of skill in the art to practice some embodiments of the invention and are merely illustrative and in no way should be construed as being limiting. In this regard, the examples should not be read as limiting the scope of the present disclosure.

Wound Electric Field (EF) and Ion Fluxes at Corneal Wounds:

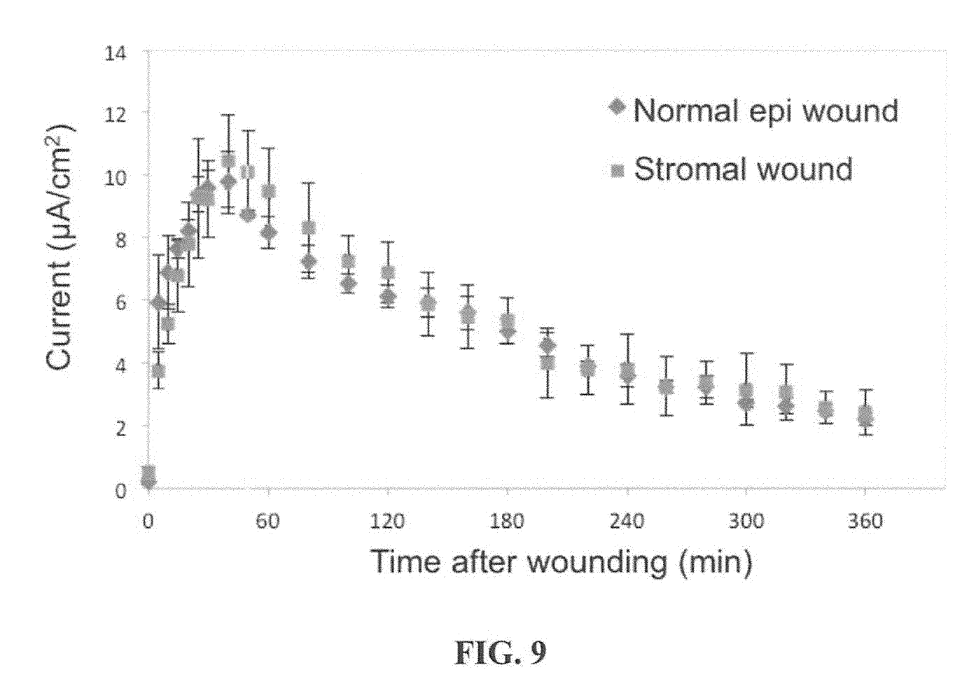

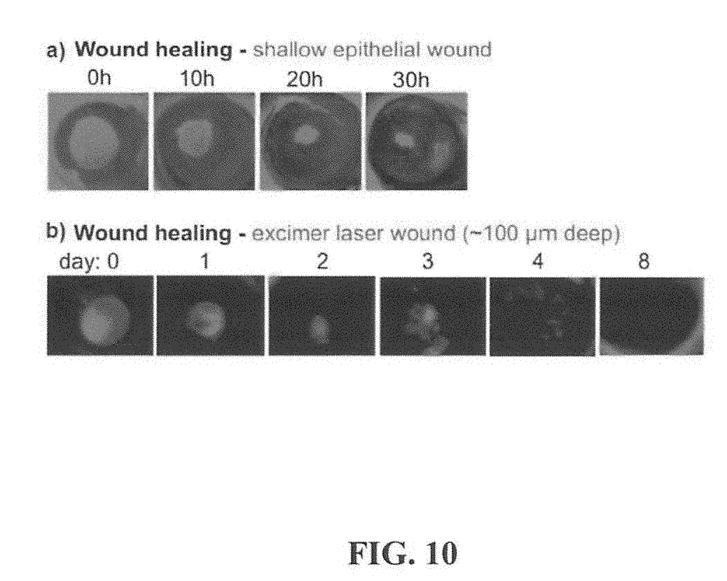

In order to understand corneal wound healing, the spatiotemporal dynamics of rat cornea wound electric currents and ion flux using a vibrating probe were characterized. In particular, measurements of deeper wounds of approximately 100 microns in depth, penetrating the stroma, were analyzed. For both shallow and deep wounds, electric current and ion flux were maximized at the wound edge, minimized at the wound center, and reduced to normal (unwounded) values at a small distance outside the wound. Wound electric current was outward (i.e., outward transient outward flux of potassium which diminished rapidly and therefore appeared to be leakage). Small fluxes of calcium and sodium were also observed. The main ion flux appeared to be a large influx of chloride ions at the wound edge. Time lapse measurements of electric current in the hours after wounding indicate that the wound electric signal was long-lasting and appears to be actively-regulated. The wound electric signal increased rapidly until approximately 40 minutes after wounding and was maintained at a high level for several hours. FIGS. 9 and 10 show time lapse data for both a shallow (normal epithelial) wound and a deep (stromal) wound. In this regard, electric signals for a shallow depth wound and a deep wound were compared. In both cases, the electric signal was significantly larger than the normal unwounded signal for at least eight hours after wounding.

Furthermore, the epithelial wounds were manually made using a biopsy punch. To replicate a more traumatic cornea wound that penetrates both the epithelium and stroma, a Nidek EC-5000 excimer laser was used. The laser settings to obtain circular wounds were 2 mm in diameter and approximately 100 μm deep. We confirmed wound depth using optical coherence tomography (OCT). Initially, corneas with laser wounds (2 mm×100 microns) showed evidence of cell damage in the epithelium outside the wound, possibly due to heat damage from the laser. The laser application was performed by first chilling eyes in a saline ice bath prior to wounding, and then applying the laser in 10 second bursts, with 5 second rest in between to minimize laser damage outside the wound. The wound edge electric signal measured from optimized stroma cornea was not significantly different from that of the shallower epithelial wound (normal: 4.19 vs. laser: 3.96 μA/cm²; P>0.7). The dynamic time course of the electric signal change in the hours after wounding was also similar to that of shallower wounds (see FIG. 9). Normal epithelial wounds 2 mm in diameter heal almost completely in 48 hours. Deep laser wounds took longer to heal than shallow epithelial wounds, but did heal 100% by day 8 (see FIG. 10).

In summary, for both shallow (about 30 microns) and deep (about 100 microns) wounds, electric current and ion flux were maximized at the wound edge, minimized at the wound center, and reduced to normal (unwounded) values a small distance outside the wound. Moreover, time-lapse measurements of electric current in the hours after wounding indicate that the wound electric signal was long-lasting and appears to be actively-regulated. The wound electric signal increased rapidly until approximately 40 minutes after wounding and was maintained at a high level for several hours. To generate reproducible deep wounds (100 microns), a laser was used and settings were optimized. Deep laser wounds take longer to heal than shallow epithelial wounds, but did heal 100% by day 8 in comparison with shallow epithelial wounds that heal quite quickly (48 hours).

Effect of Therapeutic Agent on Corneal Wounds:

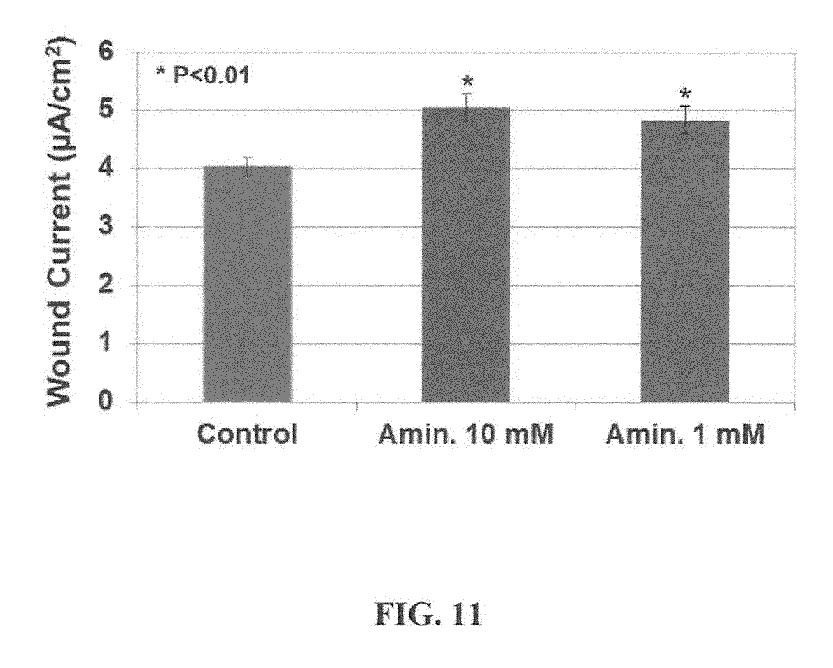

Aminophylline and ascorbic acid (10 mM) both significantly increase the cornea wound electric signal (P<0.04, P<0.03, respectively). Aminophylline also significantly increases the chloride flux at the wound (P<0.03). Both of these therapeutics also significantly enhanced cornea wound healing (P<0.01, P<0.05, respectively). Wounds 3.5 mm in diameter that normally took 48 hours or more to completely heal were healed in 30 hours in the presence of these therapeutics. To ensure that the aminophylline has the same effect on deep wounds as shallow epithelial wounds, wound electric fields were measured using the vibrating probe with and without the presence of 1 and 10 mM aminophylline. FIG. 11 illustrates the presence of aminophylline in the wound. As shown in FIG. 11, aminophylline appears to result in an increase in the wound electric field and correlates with the aminophylline concentration.

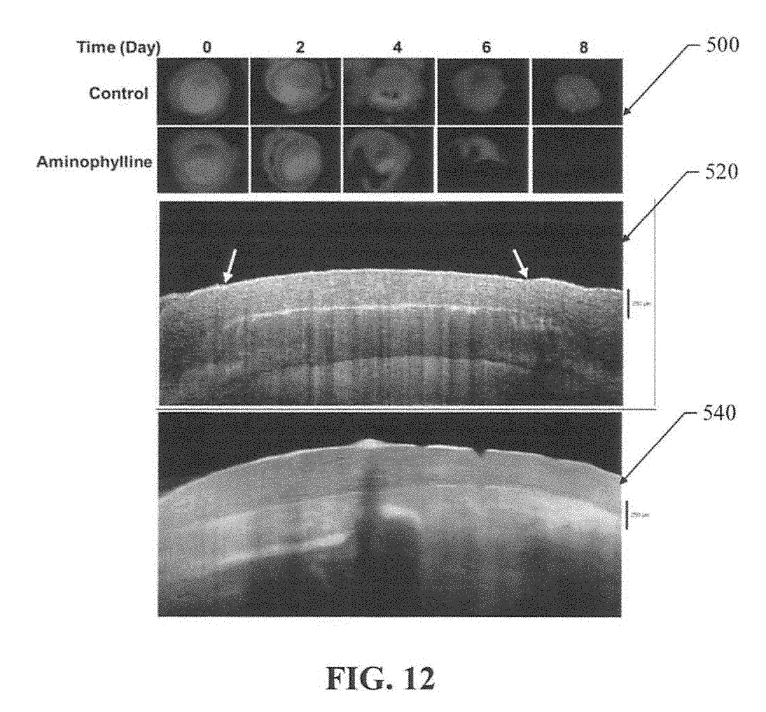

The rat model used herein shows a dynamic time course of rising and maintained electric current similar to that of a human eye. FIG. 12 illustrates wound healing of a deep wound (100 μm) without and with the presence of 1 mM aminophylline as function of time. In this regard, at 500, deep wound healing with and without the presence of aminophylline are shown. At 520, an OCT image of the control wound is shown. At 540, an OCT image with the presence of aminophylline in the wound is shown. As demonstrated by comparing the OCT images at 540 and 520, aminophylline is shown to facilitate wound healing and regeneration of an injured cornea in the deep wound faster than a control.

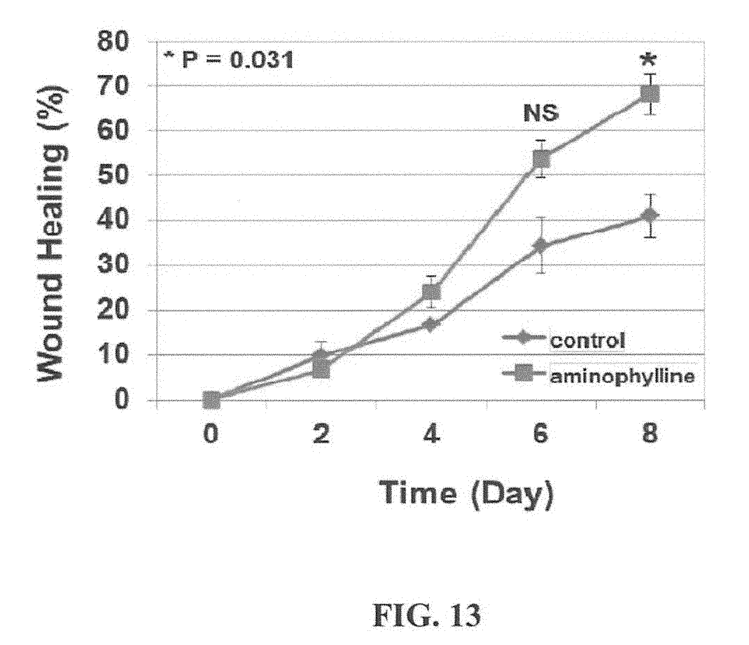

Furthermore, FIG. 13 illustrates the percentage of wound healing as a function of time for corneas treated with the bioelectric modulator aminophylline (1 mM). The untreated corneas showed only 40% wound healing in comparison with the treated corneas that showed significantly more healing (−70%). These results indicate the importance of the bioelectric modulator aminophylline in the healing of deep wounds.

Synthesis of Collagen Composites:

An example protocol used for collagen membrane fabrication is as follows: 1) collagen solution of a known concentration was placed in a metallic mold; 2) the metallic mold is placed between, but not in contact with, two metallic parallel plates; 3) a gelation process is performed using a buffer solution; 4) single layers of the collagen membrane are removed from the mold and combined into multi-layer constructs; and 5) a dehydrothermal cross-linking technique is used to increase cross-link density, resulting in collagen membranes.

This fabrication method allows tailoring of collagen membrane thickness and areal dimensions, and experimental conditions were altered to tailor fiber diameter, fiber density, thickness-normalized transparency and suture strength. This fabrication process was used in combination with Design of Experiment (DOE) to correlate the membrane fabrication parameters with resulting structure and properties. A DOE is a statistically-based method of conducting a series of experimental runs in which independent variables or factors are simultaneously varied while obtaining a desired effect or response. This approach enabled controlled tailoring of membrane morphology and properties.

Strength and Transparency of Collagen Composites:

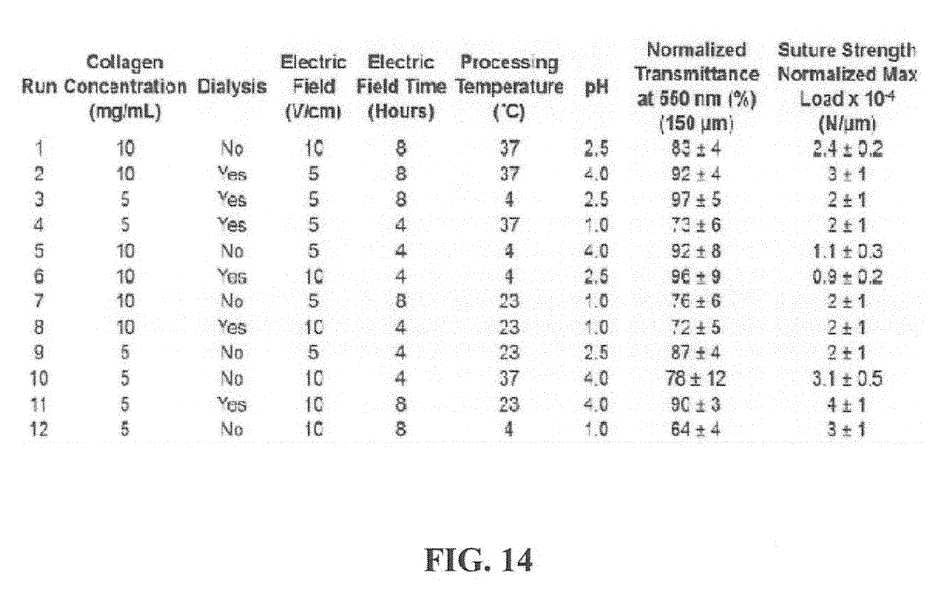

FIG. 14 illustrates factors employed in the DOE described herein. For the DOE employed herein, factors included collagen concentration (5, 10 mg/mL), dialysis (yes, no), electric field (5, 10 V/cm), electric field exposure time (4, 8 hours), processing temperature (4, 23, and 37 degrees Celsius), and collagen solution pH (1.0, 2.5, and 4.0). The design called for 12 runs. Responses measured for samples in this DOE included transmittance and suture strength. In this regard, the goal of the DOE was to produce processing-structure-properties relationships for the collagen membranes, such that materials to facilitate optimized performance may be tailored.

Transmittance was measured with a Perkin Elmer Lambda 950 UV-Vis Spectrometer. At least nine measurements were taken for each fully hydrated collagen sample and normalized for a thickness of 150 μm. Average transmittance values at 550 nm range from 64 to 96%. The transmittance values exceed those of collagen vitrigel (CV) materials and other collagen membranes reported in literature.

Fully hydrated collagen membranes were cut into 8 mm discs using a biopsy punch. Two standard 10-0 sutures (33 microns monofilaments) were pushed through the collagen discs 2 mm inside opposite edges. Sutures were placed in pneumatic grips of an Instron 5942 with a SOON load cell. In the setup, collagen membranes were suspended between the two sutures. The sutures were drawn until break and force at break was recorded. Average force at break derived from suture tests range from 0.09 to 0.4 mN/μm. The high suture strengths obtained exceed previously published data for collagen membranes. High suture strength ensures that surgeons will be able to easily manipulate the material and successfully suture the material into a wound.

The statistical analysis revealed pH (Prob.<0.0001), collagen concentration (Prob. 0.0015), electric field (Prob. 0.0160), electric field time (Prob. 0.176), and the interaction of collagen concentration and electric field (Prob. 0.0001), and collagen concentration and processing temperature (Prob. 0.0333) as influential factors for the normalized transmittance. For the maximization of transmittance, the following factors were influential: a pH of 2.5, higher collagen concentration, higher electric field strength, and longer electric field time.

The statistical analysis revealed that collagen concentration (Prob.<0.0001), pH (Prob. 0.0153), and the interaction of collagen concentration and pH (Prob. 0.0385) as influential factors for the suture strength (normalized load). Suture strength is dependent on collagen concentration, processing temperature and pH. For the maximization of suture strength, lower collagen concentration, higher processing temperature, and a pH of 2.5 were influential.

Composite Configurations:

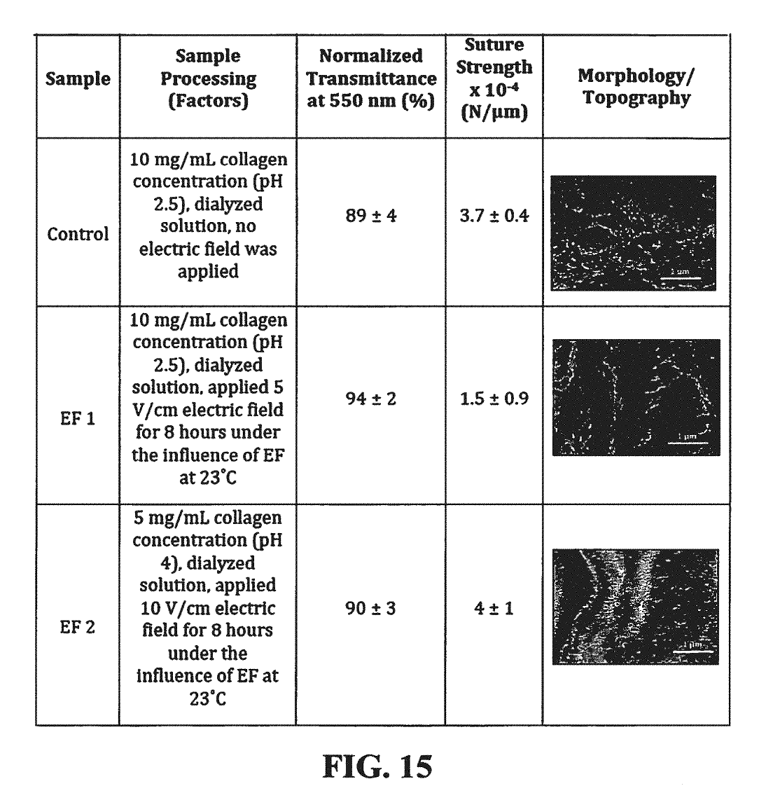

The collagen membranes selected for the remainder of the project were chosen with a goal of balancing optical and mechanical properties. FIG. 15 summarizes the down selected collagen membranes. The down selection was performed taking into consideration the fact that maximizing the properties might not result in the most effective material for the application. The thresholds used for the down selection to ensure the appropriate balance of the material properties were transparency of 75% (to maintain vision) and suture strength (corresponding to a break point of at least 0.2 mN/μm) to ensure ease of application and integrity of wound closure. All the selected materials met and exceeded the thresholds for the optical and mechanical properties. Moreover, the morphology and topography was taken in consideration for the down selection.

The elastic modulus of this subset of collagen membranes with different morphologies were measured using atomic force microscopy (AFM). The average elastic modulus of the various collagen membranes ranged between 9.5 to 56.4 kPa. Compared with the elastic modulus for different corneal layers, 0.38 kPa in posterior stroma to 11.7 kPa in Descemet's membrane, the non-aligned collagen membrane (NA) had an elastic modulus close to the Descement's membrane. The materials made under the influence of an electric field (EF1 and EF2) had elastic moduli that exceed the elastic modulus for corneal layers.

Based on findings, the NA collagen membrane was selected since it exhibited the most optimal balance between optical properties (89% transparency) and mechanical properties (3.7×10-4 N/μm suture strength, 9.5 kPa elastic modulus) for this application.

Cornea Wounds Electrical Signals:

The collagen membranes were rehydrated with a saline solution prior to in vitro experiments. To characterize the rehydration dynamics, OCT images were collected throughout the rehydration process. Collagen membranes appeared to reach maximum thickness after 24 hours in saline. No further increase of thickness was seen if the membrane was rehydrated for more than 24 hours.

The effects of two distinct rehydrated collagen membranes (with a nominal thickness of 100 μm) on cornea wound electric signal were characterized. The collagen membranes were a non-aligned collagen membrane (NA) and electric a field-aligned collagen membrane (material made under the influence of an electric field, EF). With the collagen membrane (a 2 mm circular plug made with a biopsy punch) in place, the wound edge electric signal was slightly less than normal, but not significantly lower (normalized to percentage: normal 100%, EF 93.7%, No EF 89.8%; P>0.4). The distance the vibrating probe was from the sample being measured was shown to affect measured values. As the probe was moved away from the source of electric signal, the current detected fell exponentially.

The effect of physical presence of collagen membrane and probe distance from wound edge on the electric signal was tested. The probe was placed in measuring position as close as possible to the wound edge with the collagen membrane, and the electric signal was recorded. The collagen membrane was removed, and the electric signal increased only slightly (EF 11%, NA 14%). The probe was then moved close to the wound edge and the measured signal increased dramatically (EF 115%, NA 96%). The physical presence of the collagen membrane therefore has minimal effects on the electric signal.

For long-term collagen membrane attachment during wound healing assays, the fibrin adhesives (Evicel and Tisseel) were tested. The collagen (2 mm diameter×100 μm thick) was placed onto the laser wound (2 mm diameter×100 μm deep), and a drop of the fibrin adhesive was placed over the cornea to hold the collagen in place. Using this method, the collagen membrane was held in place for duration of the experiment (4+ hours).

The characterization of wound electric signal was performed using a 2 mm diameter, 100 μm thick collagen membrane material; this was placed on the wound. The samples used in the experimentation included: control (wound, no collagen membrane in place), non-aligned collagen membrane (control, NA) and electric field-aligned collagen membrane (material made under the influence of an electric field, EF1). A separate wound electrophysiology characterization was performed after securing the collagen membrane in place using fibrin adhesive.

Securing the collagen membrane with the fibrin adhesive did not prevent the measurement of the wound current. Therefore, application of the membrane resulted in an insignificant reduction of wound current in comparison with the control (i.e., deep wound). Any current reduction was due to the distance of the vibrating probe during the measurement. The collagen membrane does not reduce the ion transport. Therefore, monitoring of the wound healing progress using the vibrating probe was possible.

Collagen Composite Topography:

The ability to monitor the wound healing process allowed for the determination of the effects of collagen membrane topography, bioelectric modulation (i.e., use of aminophylline), and any combination of these treatments. To study the effect of the collagen membrane during the wound healing assay, a drop of the fibrin adhesive was placed over the wound to hold the collagen membrane in place, as previously described. Fluorescein dye was used to monitor the wound healing progress. When the fluorescein dye was in contact with the wound bed, a fluorescent signal was observed. No fluorescent signal or label was observed when the collagen membrane material was in contact with the fluorescein dye. Furthermore, the collagen membrane material secured with the fibrin adhesive on the wound bed does not prevent the observation of the fluorescent signal when the fluorescein dye was added.

Collagen membranes with varying topography were applied to superficial wounds to understand effects of topography on healing. In this study, four eyes were evaluated: two controls with no membrane applied, and one each of NA and EF collagen membrane applied. All four eyes healed within 48 hours. Interestingly, the eye with the NA material healed slowly in the first 24 hours. However, the same eye was able to heal within 48 hours similar to the control and EF material. Using similar camera settings, wound labeling at 24 hours was not observed for the eye with the NA material.

In contrast, collagen topography was found to have negligible effect on endogenous EF (wound electrophysiology). Deep cornea wounds (100 μm) were studied to examine whether different collagen membrane morphologies affect wound healing. There was no significant difference in the end state among control group and different types of collagen membranes. However, the collagen membranes stayed in place throughout the study, which was quite important for the in vivo evaluation.

To elucidate the effect of collagen morphology on cellular behavior, the dependence of human corneal epithelial cell (hCEpiC) migratory behavior on different collagen membranes, with and without a physiologically relevant electric field, was studied. The experiments were performed using hCEpiCs (10-15 microns) seeded on tissue culture polystyrene (control, TCPS) and three different collagen membranes with distinct morphologies, with no external field. The migratory behavior for all the material morphologies, including the control TCPS, was random without any specific direction. The statistical analysis showed no significant difference in cellular velocity or direction.

Similar experimentation was performed in combination with an external applied electric field representative of physiologically relevant electric field present during wound healing. This study was focused on understanding the role and interaction of collagen materials and electric field on the cellular migration that occurs during the wound healing process. The results showed similar cellular velocity and directionality, independent of the material. The external applied electric field, however, was able to control the cellular velocity, movement and direction. We have concluded the collagen materials do not inhibit cell migration or cell response to the relevant wound electric field.

Collagen Biocomposite Structure:

Optimization of the release of aminophylline, a bioelectric modulator with well-demonstrated effect on endogenous electric field and wound healing, studied. PLGA nanoparticles were selected to encapsulate and deliver aminophylline. PLGA is extremely biocompatible. When aminophylline is encapsulated in a PLGA shell, gradual dissolution of the shell will result in the slow release of aminophylline to the wound site.

The aminophylline loaded PLGA nanoparticles were made using process known as double emulsion solvent (DES) evaporation. First, an aqueous buffered solution of aminophylline of known concentration was prepared. Separately, a solution of PGLA and Pluronic F68 in ethyl acetate was prepared. The aminophylline solution was transferred into the PLGA solution and the mixture was briefly sonicated. During sonication, the solution was kept over ice to avoid overheating. The resulting water in oil emulsion was placed into a second aqueous buffered bath, with Pluronic F68. The mixture was again briefly sonicated. The resulting water/oil/water emulsion was placed into a rotary evaporator to remove ethyl acetate. Following solvent removal, the solution was centrifuged. Effluent was discarded and particles were re-dispersed in deionized water. This washing procedure was repeated three times. The resulting purified nanoparticle solution was flash frozen with liquid nitrogen and lyophilized to afford a dry powder consisting of nanoparticles.

In order to achieve the delivery of aminophylline, a two-component biomaterial design with both a burst and extended therapeutic release was studied. Using this approach, the collagen membrane provided a burst release and PLGA therapeutic-loaded nanoparticles will provide the extended and controlled release.

So as to determine the tailorability of the therapeutic release from the PLGA nanoparticles, two different molecular weights (7-17 kg/mol and 38-54 kg/mol) were utilized. The molecular weight of the PLGA polymer directly affects the kinetics of the erosion mechanism for the nanoparticle shell. The higher molecular weight PLGA polymers yield slower shell erosion, whereas lower molecular weight polymer yield a faster erosion process. The speed of the PLGA erosion was directly related to how quickly the therapeutic was released from the nanoparticles. The ability to easily alter the therapeutic release profile by changing the polymer molecular weight represents a means to tailor the material for the treatment of ocular injuries.

As expected and regardless of morphology, the collagen membranes provided one hour burst aminophylline release via passive diffusion. This was expected due to the hydrophilic nature of the therapeutic, lack of covalent bonding to the collagen, and thin dimensions of the membranes.

On the contrary, the PLGA nanoparticles have an encapsulation efficiency of one percent for the hydrophilic therapeutic aminophylline. This was determined experimentally using liquid chromatography-mass spectroscopy (LC-MS). PLGA nanoparticles provided a longer aminophylline release, lasting two days. The release rate was independent of the PLGA molecular weight, within the range investigated; indicating that the gradual dissolution of the polymer shell was not a significant influential factor for the release of a hydrophilic therapeutic.

As such, the dominant mechanism governing release of hydrophilic aminophylline from a porous hydrophobic PLGA shell was concentration dependent.

The synthesis, purification and lyophilization protocols for the preparation of the PLGA nanoparticles were optimized to yield consistent nanoparticles with similar size and polydispersity. Fourier transform infrared spectroscopy (FTIR) analysis was used to confirm high purity of the final purified product (i.e., PLGA nanoparticles) where the surfactant or surfactant traces were not present. To extend the shelf life of the PLGA nanoparticles, lyophilization was employed without altering the physical properties (i.e., particle size and polydispersity) of the nanoparticles. The storage of the nanoparticles in water will result in the diffusion of the aminophylline, however lyophilization allows the therapeutic to stay encapsulated in the nanoparticle.

To effectively heal corneal injuries, the aminophylline loaded PLGA nanoparticles were integrated into the aminophylline loaded collagen membranes, resulting in a system with both a burst release and a sustained therapeutic release. The integration of the PLGA nanoparticles into the collagen membrane required the covalent functionalization of the nanoparticles into the membrane to ensure a robust dual system. There were several possible strategies for achieving functionalization of these individual components that were considered, including physical cross-linking, physical chemical crosslinking relying on irradiation by ultra-violet (UV) light or chemical techniques, and formation of ionic bonds. Crosslinking using UV irradiation is a practical and scalable approach in which the amino (—NH2) and carboxyl (—COOH) functional groups in collagen and PLGA can be exploited to ensure the covalent functionalization. System integration, where the PLGA nanoparticles loaded with a fluorescent dye (1 1′-diethyl-2 2′-carbocyanine iodide) was used to facilitate the visualization of the nanoparticles. The protocol used required the use of lyophilized PLGA nanoparticles. These were suspended into 0.32wt % Irgacure 2959 solution at a concentration of 100 mg/mL. The re-dispersion of the lyophilized PLGA nanoparticles in aqueous solution resulted in a well dispersed bluish suspension (indicative of a nanosuspension). Subsequently, a known amount was pipetted on the collagen membrane and allowed to absorb. Following, the loaded collagen membrane was exposed to UVA (369 nm) for 30 minutes (reaction time). After the reaction, the collagen membranes were rinsed with water (quick, 2, and 5 minutes). Fluorescent microscopy was used to visualize the incorporation of the nanoparticles into the collagen membrane.

Additionally, the dual system was characterized using scanning electron microscopy (SEM) to visualize the incorporation of the individual components. The nanoparticles were covalently incorporated into the collagen membrane as seen in the top view SEM images. Fluorescent microscopy was used to visualize and verify the incorporation of the nanoparticles into the collagen membranes over time. The results showed the stability of the dual system and the long lasting incorporation of the nanoparticles, critical for the in vivo evaluation.

The surgery procedure was optimized using a rabbit model for the in vivo evaluation. The surgical procedure included making a wound to the corneal tissue to remove the epithelium/stroma (wound size 4 mm). Then, a slit was made in the stroma all the way around to give a total diameter of 8 mm. Finally, the collagen membrane (8 mm) was placed on the wound and the edges slipped into the slit to hold it in place. In the studies, the following time points were studied, baseline, surgery (day 0), and day 1, 2, 3, 4, 5, 6, and 7 to monitor the epithelial wound healing using fluorescein dye. Additionally, the following time points were studied, baseline, surgery (day 0), and day 1, 7, 14, 21, and 28 to monitor stromal wound healing using OCT and histology at the end of the study (day 28).

The epithelium wound healing analysis using fluorescein dye revealed increases in healing rate in the presence of a collagen membrane. For the control rabbits (i.e., no collagen membrane), the epithelium healed completely after seven days. On the other hand, the experimental rabbits with a NA collagen membrane healed completely after four or five days. It is believed that the migration of the epithelial cells was enhanced in the experimental group since the collagen membrane served as a scaffold.

The stromal wound healing was monitored using OCT and revealed that the collagen membranes were in contact with the wound bed. The collagen membrane stayed in place throughout the entire study, and after 28 days post-surgery, the integration of the collagen membrane with stroma is observable. Additionally, we have measured the central corneal thickness and revealed thicker central corneal thicknesses for the eye with NA collagen membrane implanted in comparison with the control (i.e. no implant). This indicates the collagen membrane can support the structure of the cornea and remodeling of the stroma. Moreover, the collagen membrane reduces the formation of stromal haze in comparison with no implant.

Hematoxylin and eosin (H&E) staining was used to visualize the integration of the collagen membrane post-surgery at day 28. H&E staining was used to visualize the stromal wound healing with implanted collagen membrane. The wounded corneas with transplanted collagen membrane had well-organized epithelium without inflammatory response or neovascularization. However, these epithelia were 2 to 4 layers thinner in comparison with the native cornea. Fibroblasts were found attached to the implanted collagen membrane, supported cell migration and re-construction of stroma. Also, more fibroblasts in the wounded stroma were observed in comparison with the native stroma. Furthermore, the collagen membrane integration with the stroma was visible at 28 days post-surgery.

The in vivo evaluation has revealed increases in the healing rate of deep corneal wounds in a rabbit model. Additionally, the collagen membrane served as a scaffold for regenerating well-organized epithelium without inflammatory response or neovascularization. This collagen material supported cell migration and re-construction of stroma and has the ability to integrate with the stroma.

Thus, in accordance with example embodiments herein, a biomaterial implant may be provided. The biomaterial implant may include a collagen membrane and a plurality of nanoparticles embedded in the collagen membrane. Each nanoparticle may include a polymer shell and a therapeutic agent encapsulated by the polymer shell. The therapeutic agent may be configured to treat an injury, disease, or wound.

In some embodiments, the features described above may be augmented or modified, or additional features may be added. These augmentations, modifications, and additions may be optional and may be provided in any combination. Thus, although some example modifications, augmentations and additions are listed below, it should be appreciated that any of the modifications, augmentations and additions could be implemented individually or in combination with one or more, or even all of the other modifications, augmentations and additions that are listed. As such, for example, incorporating a plurality of nanoparticles into the cross-linked collagen membrane may include covalently bonding the plurality of nanoparticles to the cross-linked collagen membrane. Alternatively or additionally, the method may further include rehydrating the cross-linked collagen membrane prior to incorporating the plurality of nanoparticles. Alternatively or additionally, the method may further include performing a chemical cross-linking reaction on the cross-linked collagen membrane to form a double cross-linked collagen membrane and rehydrating the double cross-linked collagen membrane prior to incorporating the plurality of nanoparticles. Alternatively or additionally, the acidic collagen solution may have a collagen concentration from about 5 mg/ml to about 20 mg/ml. Alternatively or additionally, the polymer shell may be a poly(lactic-co-glycolic acid) (PLGA) shell. Alternatively or additionally, the bio-active therapeutic agent may be aminophylline. Alternatively or additionally, the cross-linked collagen membrane may have a thickness from about 100 microns to about 600 microns. Alternatively or additionally, applying the influence of the electric field to the acidic collagen solution positioned between the metal plates may include applying a voltage from about 1 V to about 15 V. Alternatively or additionally, the collagen membrane may include a plurality of nanofibers that are substantially aligned, and each of the nanofibers may have a fiber diameter of about 20 nanometers to about 300 nanometers. Additionally or alternatively, the collagen membrane may have a thickness of about 100 microns to about 600 microns. Additionally or alternatively, the collagen membrane may have a suture strength of about 0.09 mN/micron to 0.4 mN/micron. Additionally or alternatively, the polymer shell may have a degradation profile configured to control a release of the bio-active therapeutic agent through the polymer shell out of the collagen membrane to the injury over a predetermined period of time. Additionally or alternatively, the bio-active therapeutic agent may be a hydrophilic bio-active therapeutic agent and each of the polymer shells may be a hydrophobic polymer shell, and the degradation profile may include an initial bolus release phase and then a slow release phase, the initial bolus release phase originating from osmotic pumping of the hydrophilic bio-active therapeutic agent through the collagen membrane. Additionally or alternatively, the polymer shell may be a poly(lactic-co-glycolic acid) (PLGA) shell. Additionally or alternatively, the bio-active therapeutic agent may be aminophylline. Additionally or alternatively, the collagen membrane may have a transparency greater than 75%. Additionally or alternatively, the therapeutic agent may be further configured to increase a wound electric signal of the injury thereby increasing a healing rate of the injury, where in order to increase the wound electric signal, the bio-active therapeutic agent is configured to increase cAMP levels thereby enhancing Cl⁻ pumping to the injury.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Moreover, although the foregoing descriptions and the associated drawings describe exemplary embodiments in the context of certain exemplary combinations of elements and/or functions, it should be appreciated that different combinations of elements and/or functions may be provided by alternative embodiments without departing from the scope of the appended claims. In this regard, for example, different combinations of elements and/or functions than those explicitly described above are also contemplated as may be set forth in some of the appended claims. In cases where advantages, benefits or solutions to problems are described herein, it should be appreciated that such advantages, benefits and/or solutions may be applicable to some example embodiments, but not necessarily all example embodiments. Thus, any advantages, benefits or solutions described herein should not be thought of as being critical, required or essential to all embodiments or to that which is claimed herein. Although specific terms are employed herein, they are used in a generic and descriptive sense only and not for purposes of limitation.

That which is claimed:

1. A biomaterial implant comprising:
a collagen membrane; and
a plurality of nanoparticles embedded in the collagen membrane,
wherein at least one nanoparticle of the plurality of nanoparticles includes a polymer shell and a bio-active therapeutic agent encapsulated by the polymer shell.

2. The biomaterial implant of claim 1, wherein the collagen membrane comprises a plurality of nanofibers that are substantially aligned in a first direction in the collagen membrane, and wherein the nanofibers each have a fiber diameter of about 20 nanometers to about 300 nanometers.

3. The biomaterial implant of claim 1, wherein the collagen membrane has a thickness of about 100 microns to about 600 microns.

4. The biomaterial implant of claim 1, wherein the collagen membrane is configured to be sutured to tissue and has a suture strength of about 0.09 mN/micron to 0.4 mN/micron.

5. The biomaterial implant of claim 1, wherein the polymer shell has a degradation profile configured to control a release of the bio-active therapeutic agent through the polymer shell over a predetermined period of time.

6. The biomaterial implant of claim 5, wherein the bio-active therapeutic agent is a hydrophilic bio-active therapeutic agent and the polymer shell is a hydrophobic polymer shell, and wherein the degradation profile comprises an initial bolus release phase and then a slow release phase, the initial bolus release phase resulting from osmotic pumping of the hydrophilic bio-active therapeutic agent through the collagen membrane.

7. The biomaterial implant of claim 1, wherein the polymer shell is a poly(lactic-co-glycolic acid) (PLGA) shell.

8. The biomaterial implant of claim 7, wherein the bio-active therapeutic agent is aminophylline.

9. The biomaterial implant of claim 1, wherein the collagen membrane has a transparency greater than 75%.

10. The biomaterial implant of claim 1, wherein the bio-active therapeutic agent is further configured to increase a wound electric signal of tissue by increasing cyclic adenosine monophosphate (cAMP) levels of the tissue.

11. A method for preparing a biomaterial implant, the method comprising:
applying an electric field influence to an acidic collagen solution positioned between metal plates;
adding a buffer solution to the acidic collagen solution to form a collagen gel;
assembling a plurality of collagen gel layers;
performing a dehydrothermal cross-link on the plurality of collagen gel layers to form a cross-linked collagen membrane; and
incorporating a plurality of nanoparticles into the cross-linked collagen membrane, wherein at least one nanoparticle of the plurality of nanoparticles comprises a polymer shell and a bio-active therapeutic agent encapsulated by the polymer shell.

12. The method of claim 11, wherein incorporating a plurality of nanoparticles into the cross-linked collagen membrane comprises covalently bonding the plurality of nanoparticles to the cross-linked collagen membrane.

13. The method of claim 11, further comprising rehydrating the cross-linked collagen membrane prior to incorporating the plurality of nanoparticles.

14. The method of claim 13, further comprising:
prior to incorporating the plurality of nanoparticles,
performing a chemical cross-linking reaction on the cross-linked collagen membrane to form a double cross-linked collagen membrane; and
rehydrating the double cross-linked collagen membrane.

15. The method of claim 11, wherein the acidic collagen solution has a collagen concentration from about 5 mg/ml collagen to about 20 mg/ml collagen.

16. The method of claim 11, wherein the polymer shell is a poly(lactic-co-glycolic acid) (PLGA) shell.

17. The method of claim 16, wherein the bio-active therapeutic agent is aminophylline.

18. The method of claim 11, wherein the cross-linked collagen membrane comprises a thickness from about 100 microns to about 600 microns.

19. The method of claim 11, wherein applying the electric field influence to the acidic collagen solution positioned between the metal plates comprises applying a voltage from about 1 V to about 15V.

20. The method of claim 11, wherein applying the electric field influence to the acidic collagen solution positioned between the metal plates comprises applying a voltage at an electrode distance from about 0.1 cm and about 5 cm.
